(19) United States
(12) Patent Application Publication
(10) Pub. No.: US 2016/0354233 A1
(43) Pub. Date: Dec. 8, 2016

(54) Cold Compress for Injuries to the Body, Relief from Pain and Protection from Overheating

(71) Applicants: Stanley Sansone, Houston, TX (US); Dylan J. Sansone, Houston, TX (US)

(72) Inventors: Stanley Sansone, Houston, TX (US); Dylan J. Sansone, Houston, TX (US)

(21) Appl. No.: 15/242,395

(22) Filed: Aug. 19, 2016

Related U.S. Application Data
(63) Continuation-in-part of application No. 14/611,308, filed on Feb. 2, 2015.

(51) Int. Cl. A61F 7/10; A61F 5/055; A61F 7/02; A61F 5/02

(52) U.S. Cl. CPC A61F 7/106; A61F 5/02; A61F 5/055; A61F 7/02; A61F 2007/108; A61F 2007/0225

(57) ABSTRACT

A method and apparatus for the treatment of injuries or pain associated with the neck, spine, lower back, sacrum or central nervous system. Apparatus can also be used to cool and relax the body. The particular embodiment utilizes a neck wrap that is activated to produce an endothermic reaction to draw heat from, or apply cold to, the neck, spine and central nervous system. A clear or transparent layer is also used to help with visual inspection. In another embodiment a neck wrap contains ice cubes to draw heat from, or apply cold to, the neck, spine and central nervous system. Layers are used to accomplish different applications and colors are used to associate with selected sport teams colors or camouflaged as may be required in a battlefield environment. Another embodiment utilizes a similar device and design for a lower back wrap.

### FIELD OF THE INVENTION

This invention relates generally to a method and apparatus for the treatment of injuries or ailments of the body including but not limited to neck and spinal injury, bleeding, concussion, migraine, seizures, heat stroke or overheating through the application of accessible cold therapy. It is the intention of the current invention to be used or deployed immediately after sports injuries, injuries on the battlefield, injuries resulting from automobile accidents or other events that result in injury or pain to the neck, spine or central nervous system. It is well known that the quick application of cold treatments reduces swelling or reduces pain after events such as injuries to the body or parts of the body, in particular, to the head, neck and spine. The timely deployment of a form fitting cold therapy compress device reduces the effects of an injury and in many instances arrest the injury from worsening. In addition, reoccurring medical conditions such as migraines, seizures or heat prostration will also benefit from a timely application or deployment of a neck and spinal cold compress. It is another intention of the current invention to be used, or deployed, to cool the body after exercise or in hot environments for long periods of time through the application of insulation and radiant barriers.

### BACKGROUND OF THE INVENTION

Cryotherapeutic or cold therapy helps reduce pain, arrest swelling, decrease the hyperactivity of the central nervous system and decreases vasodilation during bleeding. Cryotherapeutic or cold therapy also helps guard against heat exhaustion, over heating and heat stroke. Advantages of emergency application of cold therapy are well known but not often used because it is difficult to carry ice around and/or the time needed to freeze water or materials for emergency use is time prohibitive. As an example, Cumming et al., U.S. Pat. No. 8,262,601 calls for ice to be inserted into a helmet.

Cryotherapeutic application during emergency situations often times may be the application of an instant ice pack. An instant ice pack is a containment pack consisting of a small bag of water and other materials, such as ammonium nitrate, calcium ammonium nitrate, urea, or others. An endothermic reaction is commenced when the water bag in the pack is broken which facilitates the mixing with or dissolving of these other materials in the pack. Such instant cold packs are square or rectangular or some other flat shape. These flat shapes are difficult to attach to the neck and spinal area and are difficult to put on and remove, often times requiring long bandages to wrap around the neck and spine. Form fitting devices for the neck and spinal are often times not available during emergency situations.

Immediately after an injury to the body, especially the neck and spine, an absorbent layer or gauze pad may be placed on the neck and spine to arrest the bleeding. This requires holding the gauze in place, which is problematic for attending emergency personnel. Wrapping a bandage around the neck and spine to secure the gauze pad is also problematic

Oftentimes a patient's neck and spine is exposed to the elements and application of any cold treatment absorbs heat from the environment instead of the patient's neck and spine.

### BRIEF DESCRIPTION OF THE DRAWINGS

FIG. 1 shows the exterior and outer layer of Neck and Spinal Cold Compress and the direction it wraps around the neck.

FIG. 2A shows the interior and inner most layer (surface closest the body) of Neck and Spinal Cold Compress. Further showing specially shaped containment packet filled with refrigerant material or materials capable of producing an endothermic reaction.

FIG. 2B shows the interior and inner most layer of Neck and Spinal Cold Compress with more than one containment packet filled with refrigerant material or materials capable of producing an endothermic reaction. FIG. 2B also shows a design for additional containment packets for the lower portion of the head in the neck and spinal cold compress. It should be recognized that a larger version of the cold compress is designed for application to the lower back, lumbar and sacrum.

FIG. 3 shows a cross sectional view of a basic emergency Neck and Spinal Cold Compress.

FIG. 4 shows a cross sectional view of FIG. 4 shows cross sectional view of Neck and Spinal Cold Compress with additional layers.

### SUMMARY OF INVENTION

The present invention addresses the timely deployment, as in an emergency application, of cryotherapeutic or cold therapeutic treatment for the body, specifically the neck and spine. An emergency neck and spinal covering cold pack reduces pain, arrest swelling, decreases the hyperactivity of the central nervous system, and decreases vasodilation during bleeding and other injuries to the neck and spine

It is the intention of the present invention to supply a form fitting neck and spinal covering secured to the neck and spine to aid in the timely stabilization and immobilization of the head and neck as in a cervical spine protocol. Said cervical spine protocol positions the neck and spinal covering to limit movement and prevent further injury caused by careless movements of the head, neck and upper spine.

In addition it is the intention of the present invention to provide an absorbent and non-stick layer fitted to the neck and spinal to absorb bleeding or other liquids appearing around the neck and spinal.

In addition it is the intention of the present invention to provide a neck and spinal covering to cool the body down when overheated or to withstand hot thermal conditions and environments.

In addition it is the intention of the present invention to provide a neck and spinal covering to cool the body down when overheated or to withstand hot thermal conditions and environments for long periods of time through the application of layers.

In addition it is the intention of the present invention to provide an insulating layer. Said insulating layer provides some physical and thermal protection for the neck and spine. Said insulating layer limits the heat from the environment thus increasing the efficiency of the refrigerant materials to deliver cold to the body, specifically the neck and spine.

In addition it is the intention of the present invention to provide a radiant barrier or layer. Said radiant barrier or layer provides some physical and thermal protection for the neck and spine. Said radiant barrier or layer limits the heat transfer by thermal radiation from the environment thus increasing the efficiency of the refrigerant materials to deliver cold to the body, specifically the neck and spine.

In addition it is the intention of the present invention to provide a porous cushioning material layer. Said porous cushioning material layer provides some physical and thermal protection for the neck and spine. Said porous cushioning material layer protects the individual wearing comfort and slow release of cold from the refrigerant materials.

In addition it is the intention of the present invention to provide cold therapy or application to a selected portion of the spine.

In addition it is the intention of the present invention to provide cold therapy or application to a selected portion of the hip and lumbar portions of the body.

In addition it is the intention of the present invention to provide clear to semi-clear materials neck and spine covering to help with observation of the neck and spine. For example said clear or transparent materials can be used to observe bleeding or other liquids appearing around the neck and spine.

### DETAILED DESCRIPTION OF THE PREFERRED EMBODIMENTS

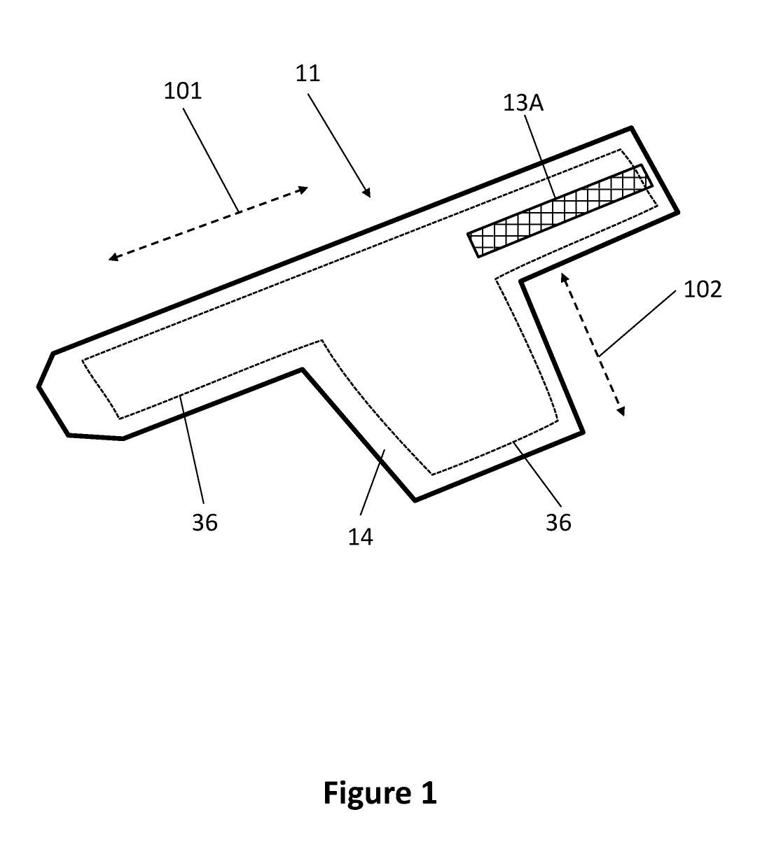

Referring to FIG. 1, Neck and spinal cold compress 11 covers the neck and a selected portion of the spine. 101 shows the direction the cold compress wraps around the neck. 102 shows the length in the perpendicular direction of the selected coverage of the spine. The length may vary depending on the amount of selected coverage. The length may run along the length of the spine and may be used in conjunction with a back or stable board. The length may also be minimal such that the neck is the only coverage as in a collar. Outer most layer 14 is the layer seen by others or when the patient looks in a mirror. Said outer most layer 14 may be of a selected color that is used to associate with sports team colors or camouflaged as may be required in a battlefield environment. Other selected colors may include colors associated with hazard or EMS related activities, be associated with a particular activities or organizations. Said selected color may be clear material which functionally allows for observation of the underlying body part. FIG. 1 shows outline of enclosed measured volume 36. Enclosed measured volume 36 is a containment pack or packet filled with refrigerant material or materials capable of producing an endothermic reaction upon activation as in an instant cold pack. Said enclosed measured volume 36 outline are shown as dashed lines and may contain more than one containment packet. Other refrigerant materials may include reusable refrigerant including but not limited to ice cubes, gel cubes or packs, hydratable ice cubes or hydratable cold packs. Fastener 13A, a single component of the fastener system, is attached to outer most layer 14 of the Neck and spinal cold compress 11.and is used for securing the apparatus to the neck and spine in a variety of configurations. Said fastener 13A as shown in FIG. 1 may be a hook type or loop type, consisting of one component of hook and loop fastening system. Adjustable fastener 13A is longer than wider such that it provides the ability to adjust for size and position on the body. This aids to gently secure neck and spinal cold compress 11 to patient's neck and spine and can be used to secure or immobilize C-spine.

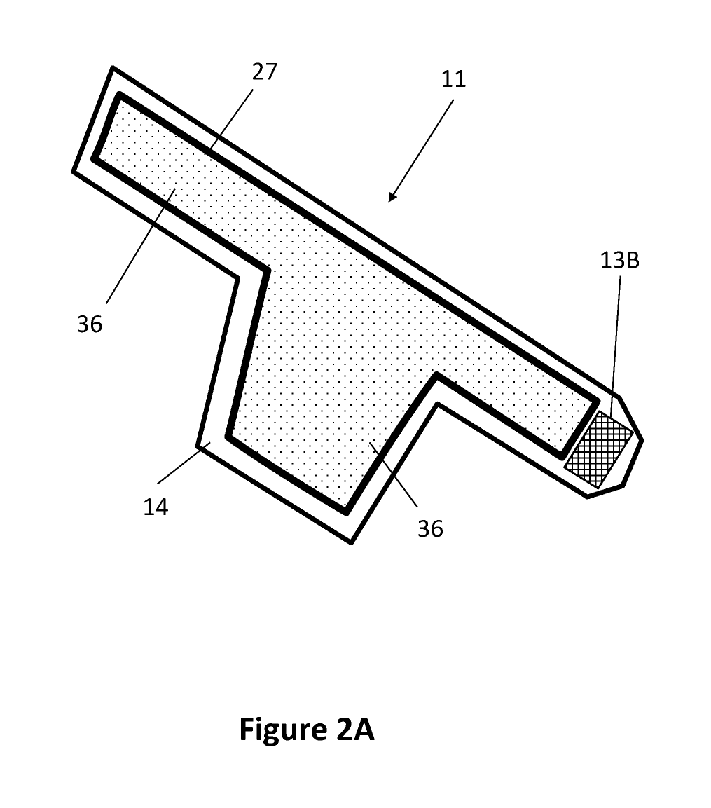

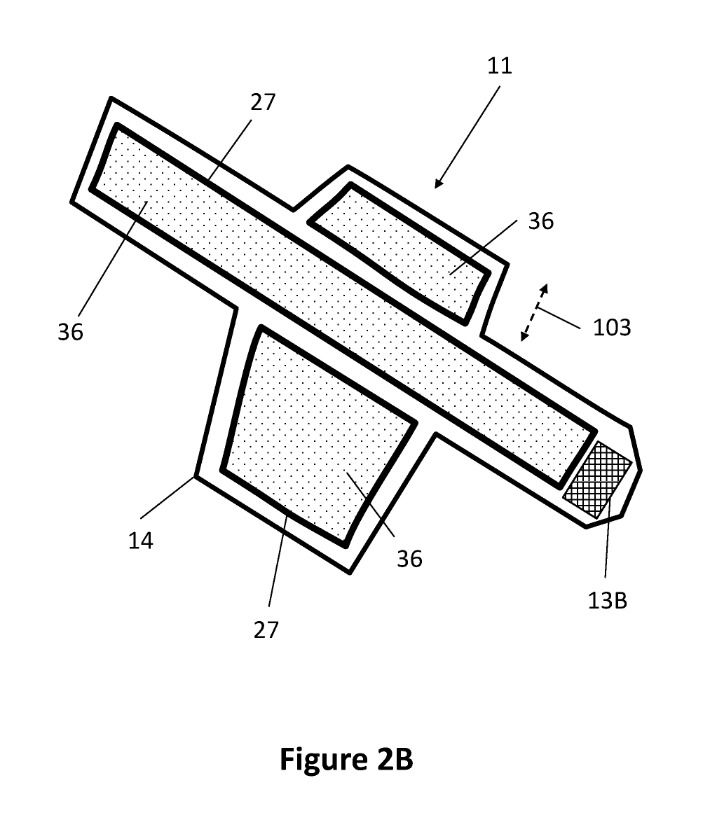

FIG. 2A shows the interior and inner most layer of Neck and Spinal Cold Compress 11. Enclosed measured volume 36 is a containment packet filled with refrigerant material. Said refrigerant material may consist of materials capable of producing an endothermic reaction upon activation as in an instant cold pack. Said enclosed measured volume 36 is shown as dashed lines. Inner most layer 14 is the layer seen by others or when the wearer looks in a mirror. Said outer most layer 14 may be of a particular color that is used to associate with selected color, sports team colors or camouflaged as may be required in a battlefield environment. Said selected color may include colors associated with hazard or EMS related activities. Said selected color may be clear or transparent material which functionally allows for observation of the underlying body part. Said selected color may be associated with a particular activities or organizations. The edges of separate enclosed measured volumes 36 may serve as seams 27 which help create the said enclosed measured volume. Fastener 138, a single component of the fastener system, is attached to inside of the Neck and spinal cold compress 11. and is used for securing the apparatus to the neck and spine in a variety of configurations. Said fastener 138 is the opposite of a selected hook or loop type shown in FIG. 1 (the front or facing side of neck and spinal cold compress). This aids to gently secure neck and spinal cold compress 11 to patient's neck and spine and can be used to secure or immobilize C-spine. Fasteners 13A (FIGS. 1) and 138 can be used to attach neck and spinal cold compress 11 to cervical spine stabilizing structure on a rigid board or backboard for emergency patient transportation.

In a preferred embodiment of a disposable apparatus shown in FIG. 2B, shows Neck and Spinal Cold Compress 11 with multiple selected and positioned enclosed measured volumes 36. In another embodiment, 103 shows direction and selected length in the perpendicular direction of additional enclosed measured volumes 36. Said additional volume in the 103 direction consists of selected coverage of cold application to the base of the head in an expanded emergency therapeutic neck and spine covering device.

A different embodiment of this invention, the device in FIG. 2B can be made and fashioned such that it straps around the waist for cold applications of the lower back, lumbar and sacrum area.

FIG. 2B also shows the Neck and Spinal Cold Compress 11 that may be reusable. In the case of a reusable compress, enclosed measured volumes 36 may be filled with water, non-freezing gel, hydratable materials or other cold holding materials. Said hydratable materials include solids that swell and retain water such that it can be frozen to provide cold therapy.

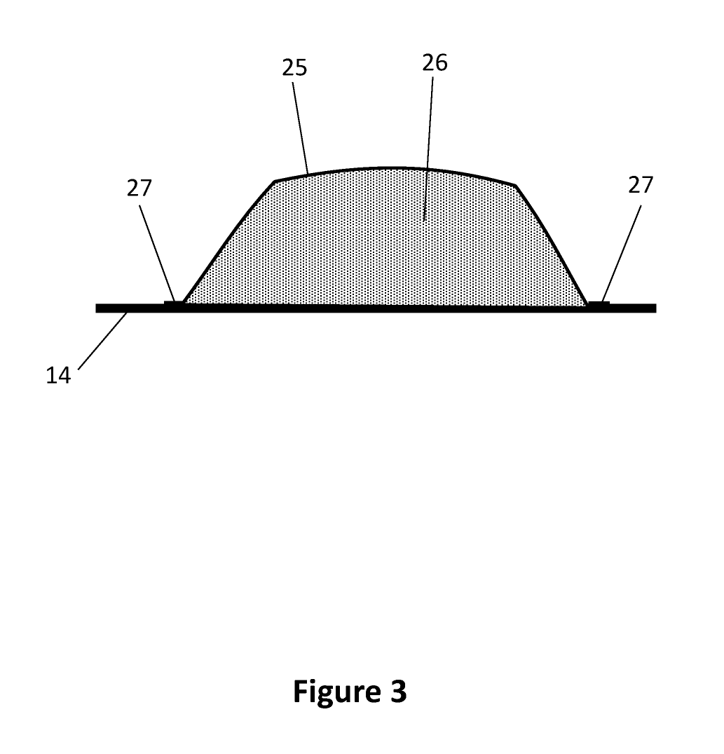

Referring to FIG. 3 showing a cross sectional view of Neck and Spinal Cold Compress 11 (FIG. 1). In the preferred embodiment outer most layer 14 provides the overall cover and a sealing surface for a portion of enclosed measured volumes 36. Inner layer 25 is attached to outer layer to form said enclosed measured volumes 36 much like a containment packet (FIG. 1). Outer most layer 14 and inner layer 25 may consist of plastic, nylon, other or some combination of materials forming said containment packet capable of holding refrigerant or endothermic materials 26. Said refrigerant or endothermic materials 26 are capable of causing an endothermic reaction upon activation. Endothermic materials for example may be ammonium nitrate and a bag of water. Other materials may be calcium ammonium nitrate, urea, or others. Activation of the reaction is a simple squeezing and bursting of the water bag. When said endothermic materials, ammonium nitrate and a bag of water, mixes an endothermic reaction results as in an instant or disposable cold pack, thus providing emergency cold treatment to a substantial portion of the neck and spine. Seam 27 is the sealed or joined part of outer most layer 14 and inner layer 25. Inner layer 25, outer most layer 14, seam 27 and endothermic materials form an instant cold pack. These multiple instant cold packs may be prepositioned with selected sizes and shapes to form the emergency Neck and Spinal Cold Compress. Other refrigerant materials may include reusable refrigerant including but not limited to ice cubes, gel cubes or packs, hydratable ice cubes or hydratable cold packs.

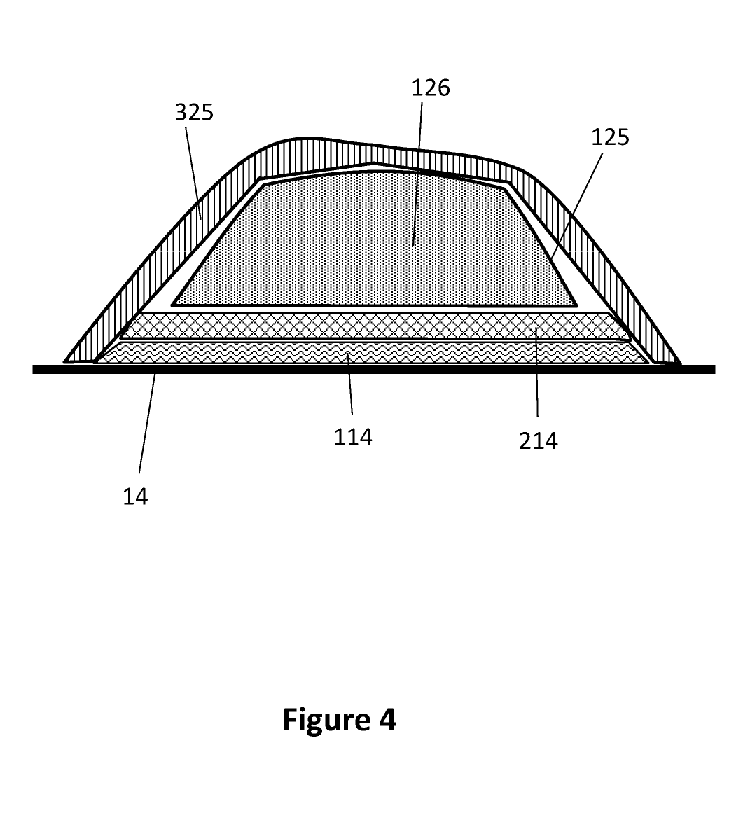

FIG. 4 shows cross sectional view perpendicular to face of the Neck and Spinal Cold Compress 11 (FIG. 1) exposing a cored refrigerant packet and multiple layers. A particular embodiment of a reusable Neck and Spinal Cold Compress where outer most layer 14 consists of a selected color shell made of non absorbent fabric such as, but not limited to, polyester, nylon, acrylic or some combination of these. Inner most layer 325 is closest to the skin and consists of a porous cushioning mesh. Said porous cushioning material could be made of PVC vinyl coated polyester mesh. Said porous cushioning material may be non-absorbent such that fluids don't get absorbed such that inner most layer 325 does not freeze into a block or become a frozen inflexible layer. In the emergency version Neck and Spinal Cold Compress said inner most layer 325 may be an absorbent porous cushioning mesh layer. Said absorbent porous cushioning mesh layer may consist of cotton, absorbent polymer fibers or absorbent particles meant to collect blood or other fluids that are on or around the patient's neck and spine. Refrigerant packet 125 is a self contained packet. In a reusable Neck and Spinal Cold Compress said refrigerant packet 125 may contain reusable refrigerant materials 126 including but not limited to ice, water, gel, hydratable materials as in hydratable cold packs. In an emergency embodiment of Neck and Spinal Cold Compress said refrigerant materials 126 may be capable of producing an endothermic reaction upon activation as in an instant cold pack. In between the refrigerant packet 125 and outer most layer 14 which is the outside of the Neck and Spinal Cold Compress 11 (FIG. 1) are additional functional layers. Insulating layer 214 and radiant barrier 114 both provide insulating capabilities to limit heat exchange between the environment and the refrigerant materials 126. Insulating layer 214 may consist of at least one layer of, but not limited to, closed cell foams, polyurethane foam, neoprene and other foams that trap air pockets and cannot soak up fluids. Other insulating layer foams may be closed cells with no air trapped in the foam but rather vacuums, providing better insulating properties. Radiant barrier 114 provides further thermal heat insulation which will reflect radiation or radiant heat. Radiant barrier 114 may use reflective material or other low emittance material. Both Insulating layer 214 and radiant barrier 114 may be used in emergency version and reusable version of the preferred embodiments. These layers, 214 and 114 may be pre bonded together for easy manufacturing.

What is claimed:

1) A method of deploying emergency cold treatment to the neck and spine comprising:
Activating at least one instant cold pack, placing said instant cold packs in a neck wrap configuration covering a substantial portion of the neck and a selected portion of the spine, securing said neck wrap to immobilize head and neck of patient.

2) A method of deploying emergency cold treatment to the neck and spine of claim 1 wherein said activating is the squeezing of instant cold pack.

3) A method of deploying emergency cold treatment to the neck and spine of claim 1 wherein said configuration are predetermined shapes and positions of instant cold packs to cover the neck and a selected portion of the spine.

4) A method of deploying emergency cold treatment to the neck and spine of claim 1 wherein said emergency cold treatment absorbs fluids around the neck and spine.

5) A method of deploying emergency cold treatment to the neck and spine of claim 1 wherein said emergency cold treatment is color selected based on a team affiliation.

6) A method of deploying emergency cold treatment to the neck and spine of claim 1 wherein said emergency cold treatment is color selected based on environment of deployment.

7) An emergency therapeutic neck and spine disposable device covering, comprising:
at least one outer layer,
attached to said outer layer are selected containment packets,
said containment packets are position to wrap around the neck and extend down the spine,
said containment packets contain materials capable to activate an endothermic reaction,
attaching devices are positioned to hold the said neck and disposable device around the neck.

8) An emergency therapeutic neck and spine disposable device covering of claim 7 wherein each internal volume has a predetermined shape and position to cover a selected portion of the spine.

9) An emergency therapeutic neck and spine disposable device covering of claim 7 wherein said outer layer is transparent.

10) An emergency therapeutic neck and spine disposable device covering of claim 7 wherein an inner layer is absorbent.

11) An emergency therapeutic neck and spine disposable device covering of claim 7 wherein inner most layer is permeable to liquids to perform function to prevent sticking to the head.

12) An emergency therapeutic neck and spine disposable device covering of claim 7 contains at least one layer of insulation.

13) An emergency therapeutic neck and spine disposable device covering of claim 7 contains a radiant barrier layer.

14) A neck and spine reusable cooling covering device comprising:
a selected colored outer layer,
a radiant layer,
an insulating layer,
a refrigerant layer, said refrigerant layer consisting of containment packets of selected shapes,
a selected porous thermal cushion mesh material, said material consists of a water resistant latticework, and
an adjustable fastener.

15) A neck and spine reusable cooling covering device of claim 14 wherein each containment packet has a predetermined shape and position to cover neck and a selected portion of the spine.

16) A neck and spine reusable cooling covering device of claim 14 wherein said outer most layer is color selected based on a team affiliation.

17) A reusable cooling covering device of claim 14 wherein said containment packets comprises cubes.

18) A reusable cooling covering device of claim 14 wherein said device is used to cool the spine, lower back and sacrum.

19) A reusable cooling covering device covering of claim 14 contains at least one layer of insulation.

20) A reusable cooling covering device covering of claim 14 contains a radiant barrier layer.
